(19) United States
(12) Patent Application Publication
(10) Pub. No.: US 2019/0059742 A1
(43) Pub. Date: Feb. 28, 2019

(54) PHYSIOLOGICAL PARAMETER SENSING SYSTEM WITH HEAT ISOLATION MECHANISM

(71) Applicant: Welch Allyn, Inc., Skaneateles Falls, NY (US)

(72) Inventors: Kenzi L. Mudge, Skaneateles, NY (US); Craig M. Meyerson, Syracuse, NY (US); Zhon Ye Chu, Syracuse, NY (US); Michael T. McMahon, Syracuse, NY (US); David E. Quinn, Auburn, NY (US)

(21) Appl. No.: 16/114,319

(22) Filed: Aug. 28, 2018

Related U.S. Application Data
(60) Provisional application No. 62/552,031, filed on Aug. 30, 2017.

(51) Int. Cl. A61B 5/01; A61B 5/024; A61B 5/1455

(52) U.S. Cl. CPC A61B 5/01; A61B 5/6833; A61B 5/14552; A61B 5/02405

(57) ABSTRACT

A physiological parameter sensing system provides a sensing device for measuring a physiological parameter of a subject. The sensing device includes various components that operate as heat sources, which can influence the operation of sensors in the device. The sensing device includes a heat isolation mechanism that thermally isolates the sensors from the heat sources.

### BACKGROUND

Wearable body sensors can be used to efficiently monitor physiological parameters, such as body temperature, in various situations. Some examples of wearable body sensors are designed to be attached to a patient body to detect physiological parameters. Wearable body sensors include various components that generate heat during operation, such as a processing device and a wireless communications device for communicating with an interrogation device. Such heat can influence the operation of the wearable body sensors and cause inaccurate readings of physiological parameters.

### SUMMARY

In general terms, the present disclosure relates to a physiological parameter sensing system with heat isolation mechanism. In one possible configuration and by non-limiting example, the system includes a heat source, and the heat isolation mechanism operates to isolate a sensor from the heat source. Various aspects are described in this disclosure, which include, but are not limited to, the following aspects.

One aspect is a physiological parameter sensing device. The sensing device includes a mounting portion and a circuit board. The mounting portion is configured to engage with a subject. The circuit board includes a processing unit, a sensing subcircuit connected to the processing unit and configured to measure a physiological parameter of the subject, and a heat isolation device connected to the processing unit and configured to thermally isolate the sensing subcircuit from the processing unit.

Another aspect is a temperature sensing patch including a mounting layer and a circuit board. The mounting layer is engagable with a subject. The circuit board includes a processing unit, a pair of thermistor pads configured to measure heat flux associated with the subject, a pair of thermistor tracks connecting the pair of thermistor pads to the processing unit, and a heat isolation device connected to the processing unit and configured to thermally isolate the pair of thermistor pads from the processing unit.

Yet another aspect is a system for monitoring a physiological parameter of a subject. The system includes a sensing device and an interrogation device. The sensing device includes a mounting portion engaged with a subject, and a circuit board including a processing unit, a sensing subcircuit connected to the processing unit and configured to measure a physiological parameter of the subject, and a heat isolation device connected to the processing unit and configured to thermally isolate the sensing subcircuit from the processing unit. The interrogation device is operable by a user and configured to establish communication with the sensing device, and receive measurement data from the sensing device.

### BRIEF DESCRIPTION OF THE DRAWINGS

FIG. 1 schematically illustrates an example system for monitoring a physiological parameter using a physiological parameter sensing device.

FIG. 2 schematically illustrates an example of the sensing device.

FIG. 3 is a schematic, exploded view of the sensing device configured as a temperature sensing patch.

FIG. 4 schematically illustrates an example arrangement of thermistors for heat flux measurement.

FIG. 5 is a block diagram of a circuit board of the sensing device.

FIG. 6 schematically illustrates an example of the circuit board.

FIG. 7 is a schematic view of a front side of the circuit board of FIG. 6.

FIG. 8 is an expanded view of the circuit board of FIG. 7.

FIG. 9 is a partial schematic view of another example circuit board.

FIG. 10 illustrates an exemplary architecture of an interrogation device.

FIG. 11 a schematic view of a rear side of the circuit board of FIG. 6.

### DETAILED DESCRIPTION

Various embodiments will be described in detail with reference to the drawings, wherein like reference numerals represent like parts and assemblies throughout the several views.

In general, the system of the present disclosure includes a physiological parameter sensing device having a sensing subcircuit configured to measure a physiological parameter of a subject. One example of the sensing subcircuit includes a thermistor. The sensing device further includes various components that can operate as heat sources, which can influence the operation of the sensing subcircuit. Examples of such heat sources include a processing unit and one or more elements associated with or interacting with an electromagnetic field generated around the sensing device. The sensing device can be provided with a heat isolation mechanism that thermally isolates the sensing subcircuit from the heat sources.

In certain examples, the heat isolation mechanism includes one or more conductive tracks that connect the sensing subcircuit with the heat sources. The conductive tracks can be configured to be thin enough to prevent heat from traveling down from the heat sources to the sensing subcircuit. In addition or alternatively, the conductive tracks can be at least partially routed to provide a long track between the heat sources and the sensing subcircuit. Such extended lengths of the conductive tracks enable reducing or preventing the thermal effect of the heat sources on the sensing subcircuit. The conductive tracks can be further configured to reduce radio interference in the sensing device. In addition, other conductive tracks in the sensing device can be similarly configured to be thin and/or have extended lengths.

In certain examples, the heat isolation mechanism includes a heat dissipation device connected to the heat sources. The heat dissipation device is configured and arranged to draw and dissipate heat from the heat sources, thereby preventing the influence of the heat sources on the sensing subcircuit.

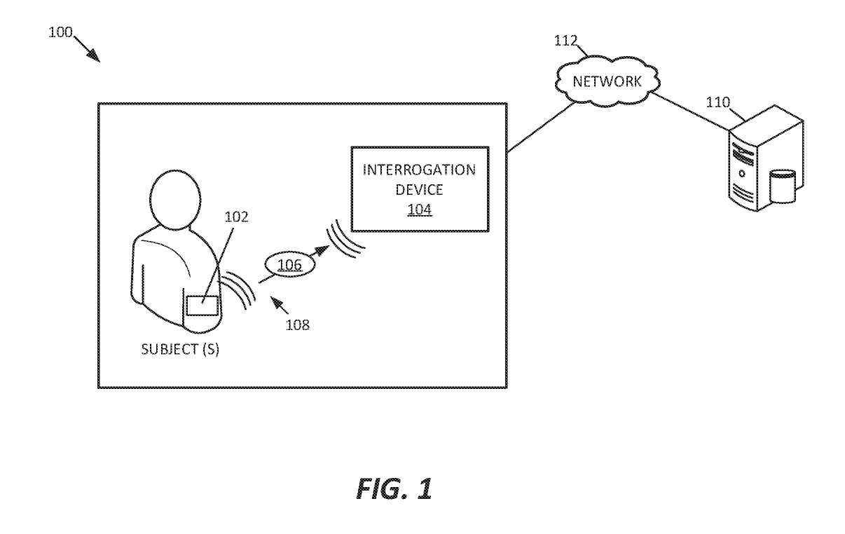

FIG. 1 schematically illustrates an example system 100 for monitoring a physiological parameter using a physiological parameter sensing device 102. The physiological parameter sensing device 102 operates to detect one or more physiological parameters of a subject S. In some embodiments, the system 100 further includes an interrogation device 104 configured to communicate with the physiological parameter sensing device 102.

The physiological parameter sensing device 102 is worn or carried by the subject S. Various methods can be used to mount the physiological parameter sensing device 102 to the subject. In one example, one or more adhesive pads or layers can be used to attach the physiological sensing device 102 (e.g., when configured as a patch) to the skin or clothing of the subject. In another example, a wrap or band can be used to mount the physiological sensing device 102 (e.g., when configured as a watch or any other body wrap or mount device) around the body limbs, head, upper body, or the like. Other fastening devices can be used in other examples.

In some examples, the sensing device 102 includes a physiological parameter sensing patch. In this document, therefore, the sensing device 102 is also referred to as a physiological parameter sensing patch, a sensing patch, a monitoring patch, a wearable body sensor, or the like.

In some examples, the sensing device 102 is removably attached to a portion of the subject's body or the subject's skin. The sensing device 102 can be worn on different locations of the subject body, such as the forehead, torso, neck, arm, leg, or other on-body locations, for different measurements. In other examples, the sensing device 102 is implanted to the subject's body. The sensing device 102 can be attached or implanted to the subject S by a healthcare practitioner when the healthcare practitioner sees the subject S. In other examples, the subject S can wear or attach the sensing device 102 on his or her own.

The sensing device 102 operates to detect one or more physiological parameters of the subject S. The subject S can also be referred to herein as a patient or person. The sensing device 102 is configured to detect one or more physiological parameters. In some examples, the sensing device 102 includes one or more sensor units. In some examples, a single sensor unit can be used to measure physiological parameters of the same type. In other examples, a plurality of sensor units can be used to measure physiological parameters of the same type. In yet other examples, the sensing device 102 includes a plurality of sensor units of different types capable of detecting different kinds of physiological parameters. The sensing device 102 transmits signals to the interrogation device 104 via a wireless communication link 108.

Physiological parameters can include vital signs, physiological measurements, and biological measurements, which can be detected from various portions of the subject's body. For example, physiological parameters include measurements of the body's basic functions, which are useful in detecting or monitoring medical problems. Examples of physiological parameters include body temperature, pulse rate (i.e., heart rate), respiration rate (i.e., breathing rate), blood pressure, blood gas, and SpO2. Body temperature can be taken in various manners, such as orally, rectally, by ear, or by skin. The pulse rate is a measurement of the heart rate, or the number of times the heart beats per minute. The pulse rate can also indicate a heart rhythm and the strength of the pulse. The pulse can be taken on different body portions where the arteries are located, such as on the side of the neck, on the side of the elbow, or at the wrist. The respiration rate is the number of breaths a person takes per minute and is used to note whether the person has any difficulty breathing. Blood pressure is the force of the pushing against the artery walls. There may be other vital signs, such as pain, Glasgow coma scale, pulse oximetry, blood glucose level, end-tidal CO₂, functional status, shortness of breath, and gait speed.

In the present disclosure, the sensing device 102 is primarily described to be capable of measuring a body temperature of the subject S. In other examples, the sensing device 102 can be configured to measure different physiological parameters, such as blood gas, SpO2, blood pressure, heart rate, and any other parameters, in addition to or in lieu of body temperature.

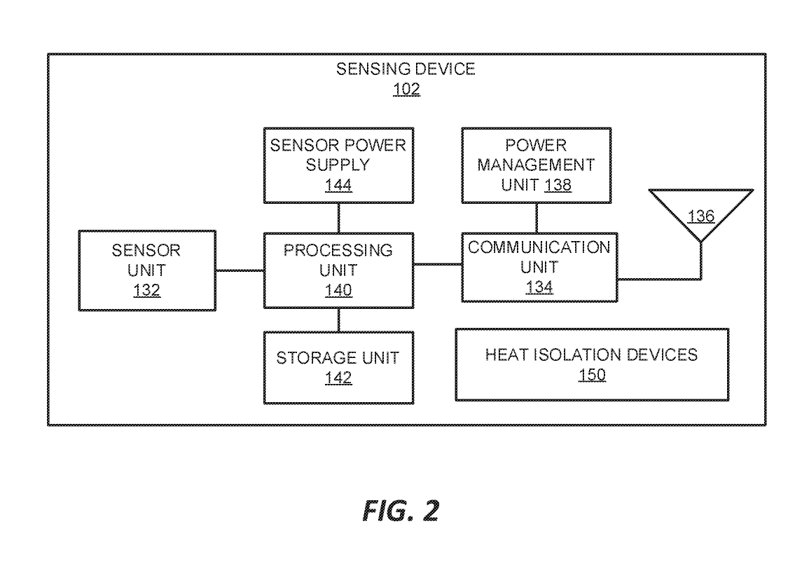

In some examples, the sensing device 102 is configured as a passive device, which does not include an independent power source, such as a battery, to supply power to the components of the sensing device 102. In this configuration, the sensing device 102 can be activated by the interrogation device 104 when the interrogation device 104 comes close to the sensing device 102 within a predetermined activation or read range. In other examples, the sensing device 102 is configured as an active device, which includes its own power supply. An example of the sensing device 102 is described and illustrated in more detail with reference to FIG. 2.

With continued reference to FIG. 1, the interrogation device 104 operates to communicate with the sensing device 102 attached to the subject S. The interrogation device 104 can receive signals from the sensing device 102 via the wireless communication link 108. In some examples, the interrogation device 104 is operable to present the data transmitted from the sensing device 102 thereon. For example, the interrogation device 104 includes a display screen and operates to present the transmitted data on the screen in a visible format. The interrogation device 104 can output the data in an audible format, and/or provide an alert in visible and/or audible manners. The interrogation device 104 can also be in communication with the data management system 110 via the network 112.

The interrogation device 104 can be used by a guardian and/or a healthcare practitioner to monitor the measurement of the sensing device 102. The guardian is a person or a group of people who is interested in the health conditions of the subject S. Examples of the guardian include a parent of the subject S, a family member of the subject S, a caregiver of the subject S, a primary physician of the subject S, and any other interested parties. The healthcare practitioner is a person who provides healthcare service to the subject S. Examples of healthcare practitioners P include primary care providers (e.g., doctors, nurse practitioners, and physician assistants), nursing care providers (e.g., nurses), specialty care providers (e.g., professionals in various specialties), and health professionals that provide preventive, curative, promotional and rehabilitative health care services. The healthcare practitioner can be an institution, company, business, and/or entity. In other examples, the interrogation device 104 can be operated by the subject S him or herself.

The interrogation device 104 can be of various types. In some examples, the interrogation device 104 is a computing device dedicated for particular sensing devices 102. In other examples, other consumer level computing devices can be used for the interrogation device 104. Such computing devices can include a mobile computing device, such as a smartphone, (e.g., an iPhone, an Android operating phone, a Blackberry, a Window operating phone, etc.); a tablet computer (e.g., an iPad), and a personal digital assistant (PDA). The interrogation device 104 can include a desktop computer, a laptop computer, and/or any other suitable devices operable to send and receive signals, store and retrieve data, and/or execute modules.

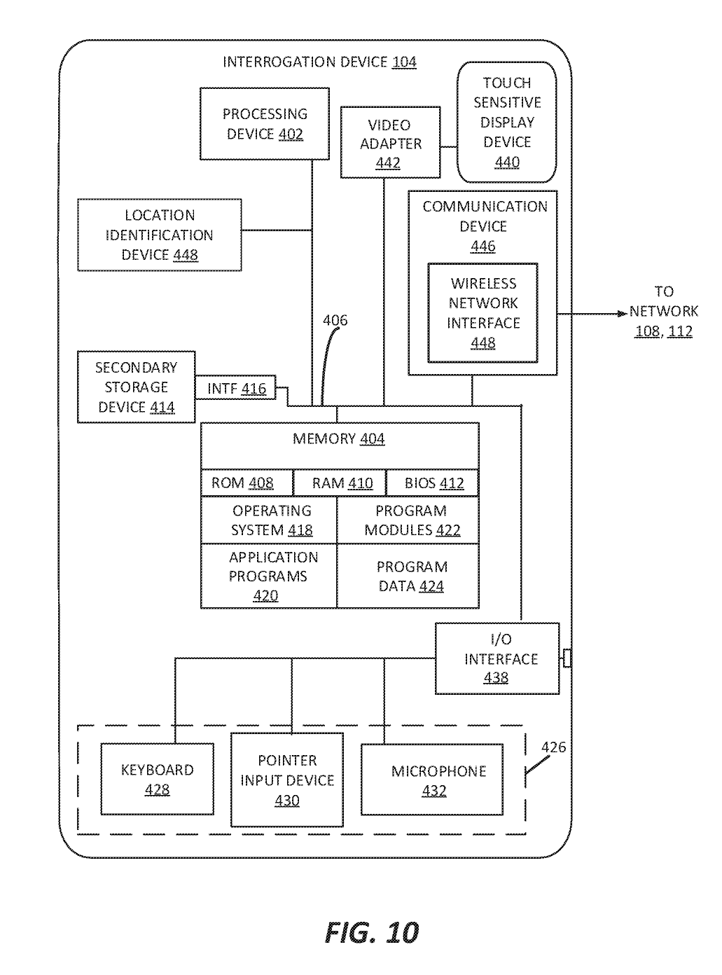

In some examples, the interrogation device 104 is configured a portable reader. Such a portable interrogation device 104 can be configured as an independent handheld device, or as a device that is connected to a movable clinical data station or equipment. As described herein, for home care, the interrogation device 104 can be various consumer mobile devices as described above. In other examples, the interrogation device 104 is mounted to a structure or device that the subject S periodically or continuously uses. For example, the interrogation device 104 is mounted to the sides or side rails of a hospital or homecare bed for a patient, such that the interrogation device 104 remains within, or easily comes into, a read range of the sensing device 102 attached to the patient's body. In yet other examples, the interrogation device 104 is incorporated into, or used with, other monitoring systems, such as Connex® Vital Signs Monitor (CVSM) available from Welch Allyn Inc., Skaneateles Falls, N.Y. An example of the interrogation device 104 is described in more detail with reference to FIG. 10.

The interrogation device 104 can receive signals from the sensing device 102 via the wireless communication link 108. In some examples, the interrogation device 104 is operable to present the data transmitted from the sensing device 102 thereon. For example, the interrogation device 104 includes a user interface (e.g., a display screen) and operates to present the transmitted data on the screen in a visible format. Alternatively, the interrogation device 104 can output the data in an audible format, and/or provide an alert in visible and/or audible manners.

The sensing device data 106 can include data stored in the sensing device 102. At least a portion of the sensing device data 106 can be transmitted to the interrogation device 104 and presented to the user of the interrogation device 104.

As illustrated in FIG. 1, the wireless communication link 108 is established between the sensing device 102 and the interrogation device 104. At least a portion of data (e.g., the sensing device data 106) stored in the sensing device 102 is wirelessly transmitted to the interrogation device 104 via the wireless communication link 108. The wireless communication link 108 can be established as short range wireless communication, such as radio frequency identification (RFID) communication, near field communication (NFC), Bluetooth communication, or Wi-Fi communication.

In some examples, the interrogation device 104 is configured as an active RFID reader and capable of communicating with the sensing device 102, which correspondingly includes a RFID device (e.g., a RFID tag). When the interrogation device 104 is brought close enough to the sensing device 102 attached to the subject S, a short-range RF communication is established between the sensing device 102 and the interrogation device 104 via electromagnetic fields so that query, authorization/authentication, and/or data interchange processes are performed between the sensing device 102 and the interrogation device 104.

In other examples, the interrogation device 104 includes a NFC interface for establishing radio communication with the sensing device 102 by bringing the interrogation device 104 into proximity to the sensing device 102 or touching the interrogation device 104 with the sensing device 102. The NFC interface can be configured in a way known in the art. The sensing device 102 is correspondingly configured to communicate with the NFC interface of the interrogation device 104. As such, the interrogation device 104 operates as an NFC reader and the sensing device 102 functions as an NFC tag.

In either of the RFID and NFC implementations, the sensing device can be passive or active. For example, the RFID- and NFC-based sensing devices may be powered by the reader's electromagnetic field. Or, the RFID- and NFC-based sensing devices may use an internal source of power, such as a battery, solar panel, or Peltier generator. Other configurations are possible.

In yet other examples, the interrogation device 104 includes a Bluetooth communication interface to establish Bluetooth wireless connection with the sensing device 102. The Bluetooth communication interface can be configured in a way known in the art. The sensing device 102 is also configured to be capable of establish Bluetooth communication with the interrogation device 104. As such, the sensing device 102 and the interrogation device 104 can be correspondingly configured to transmit data via low-power radio waves. In some examples, the Bluetooth Low Energy (BLE) wireless technology can be used.

In yet other examples, the interrogation device 104 includes a Wi-Fi communication interface to establish Wi-Fi connection with the sensing device 102. The Wi-Fi communication interface can be designed in a way known in the art. The sensing device 102 is also configured to communicate with the Wi-Fi communication interface of the interrogation device 104. As such, the sensing device 102 and the interrogation device 104 can be correspondingly configured to transmit data via radio waves. By way of non-limiting example, and as will be appreciated by those skilled in the relevant arts, Wi-Fi can be deployed in accordance with IEEE 802.11 (Wireless LAN), IEEE 802.15.4 (Low-Rate wireless PAN, such as ZigBee, WirelessHART, and MiWi), IEEE 802.22 (Wireless Regional Area Network), Wi-Fi Direct, or other standard. In some embodiments, Wi-Fi connection can be alternatively established if other connections (e.g., RFID, NFC, and Bluetooth) are not established.

In other embodiments, the wireless communication link 108 can implement other types of short-range communications, such as infrared data communication, Z-Wave, ANT+, and other suitable protocols.

Although the wireless communication link 108 is primarily described in the present disclosure, other embodiments are also possible where a wired communication link replaces the wireless communication link 108 or used together with the wireless communication link 108.

With continued reference to FIG. 1, in some examples, the system 100 is operable to communicate with a data management system 110 via a data communication network 112. The data management system 110 operates to manage the subject's personal and/or medical information, such as health conditions and other information. The data management system 110 can be operated by the healthcare practitioner and/or a healthcare service provider, such as a hospital or clinic.

Some embodiments of the data management system 110 are configured to communicate with either or both of the sensing device 102 and the interrogation device 104. For example, the interrogation device 104 and the data management system 110 are connected via the network 112 to transmit various data therebetween. In other examples, the sensing device 102 is capable of directly communicating with the data management system 110 to transmit measurement data (and other data associated with the subject S). In some examples, the data management system 110 operates to provide information that can be used to assist the subject S, the guardian and/or the healthcare practitioner to provide suitable healthcare to the subject S. In some examples, the data management system 110 includes such a computing device as described in FIG. 10. Examples of the data management system 110 include Connex® data management systems available from Welch Allyn Inc., Skaneateles Falls, N.Y.

The data communication network 112 communicates digital data between one or more computing devices, such as among the sensing device 102, the interrogation device 104, and the data management system 110. Examples of the network 112 include a local area network and a wide area network, such as the Internet. In some embodiments, the network 112 includes a wireless communication system, a wired communication system, or a combination of wireless and wired communication systems. A wired communication system can transmit data using electrical or optical signals in various possible embodiments. Wireless communication systems typically transmit signals via electromagnetic waves, such as in the form of optical signals or radio frequency (RF) signals. A wireless communication system typically includes an optical or RF transmitter for transmitting optical or RF signals, and an optical or RF receiver for receiving optical or RF signals. Examples of wireless communication systems include Wi-Fi communication devices (such as utilizing wireless routers or wireless access points), cellular communication devices (such as utilizing one or more cellular base stations), and other wireless communication devices.

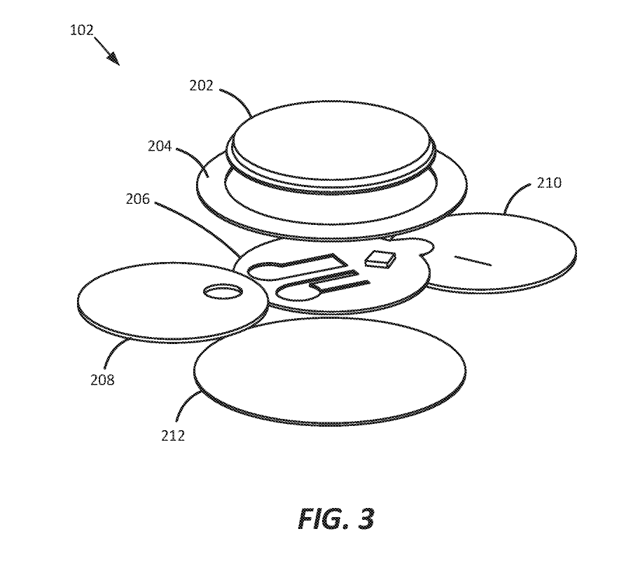

FIG. 2 schematically illustrates an example of the sensing device 102, which is used to measure physiological parameters of the subject S. In the illustrated example, the sensing device 102 includes a sensor unit 132, a communication unit 134, an antenna 136, a power management unit 138, a processing unit 140, a storage unit 142, and a sensor power supply 144. In addition, the sensing device 102 includes one or more heat isolation devices 150. In other embodiments, the sensing device 102 can include one or more components in addition to the components described above, and/or replace one or more of the components described above by different components. In some examples, the sensing device 102 is at least partially implemented in zero or more integrated circuit(s). The integrated circuit(s) can be included as part of a circuit board 206 (FIG. 3).

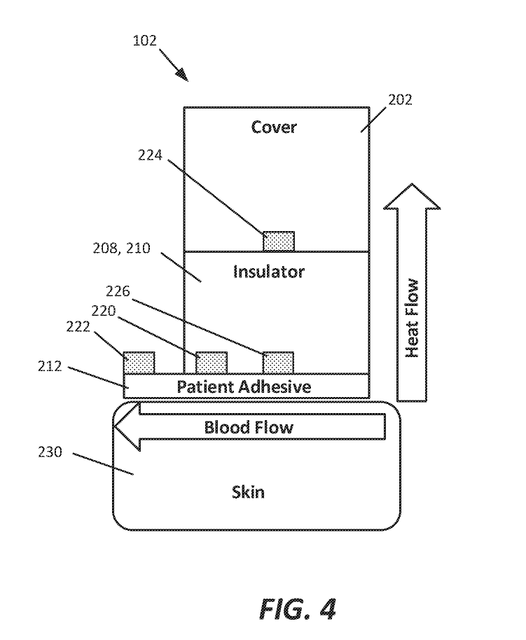

The sensor unit 132 includes one or more sensors operable to detect one or more physiological parameters. In some embodiments, each sensor is configured as a sensing subcircuit in a circuit board (such as a circuit board 206 in FIG. 4) of the sensing device 102.

In some examples, the sensor unit 132 includes one sensor for detecting one type of physiological parameters. In other examples, the sensor unit 132 includes a plurality of sensors for detecting same or different types of physiological parameters. Example sensors of the sensor unit 132 include temperature sensors, heartrate sensors, electrocardiogram (ECG) sensors, respiratory rate sensors, accelerometers, SpO2 sensors, heartrate variability sensors, galvanic skin response sensors, blood pressure sensors, blood glucose sensors, blood oxygen sensors, and any other sensors suitable for measuring physiological parameters. The sensor unit 132 can further include one or more sensors (e.g., accelerometer) for detecting the subject's activity and posture, such as whether the subject is standing, sitting, laying down, or engaged in physical activity, such as running. In some examples, the sensor unit 132 is powered by the sensor power supply 144.

As illustrated herein, where the sensing device 102 is configured as a temperature sensing patch, the sensor unit 132 includes one or more temperature sensors, such as thermistors.

The communication unit 134 operates to communicate with the interrogation device 104. In some examples, the communication unit 134 can receive signals from the interrogation device 104 via the wireless communication link 108 and transmit data (e.g., sensing device data 106) to the interrogation device 104. For example, the communication unit 134 can operate as a transponder configured to emit an identifying signal in response to an interrogating received signal from the interrogation device 104. The communication unit 134 is configured as an interface suitable for communicating with the interrogation device 104, such as near field communication (NFC), radio frequency identification (RFID), Bluetooth, Wi-Fi, and other short-range wireless communications. In other examples, the communication unit 134 is further configured to communicate with the data management system 110 and/or other computing devices via the network 112.

The antenna 136 is configured to receive and transmit a radio frequency (RF) signal. In some examples, the antenna 136 is made flat so as to be incorporated into the sensing device 102. Other configurations are also possible in other embodiments.

The power management unit (PMU) 138 operates to harvest raw RF power received via the antenna 136. In particular, an RF wave received via the antenna 136 is transmitted to the PMU 138 as a signal. The signal is used for harvesting the power and also decoded for further processes. The sensing device 102 then use the power to respond as necessary in response to the incoming signal from the interrogation device 104.

In some examples, in the communication between the sensing device 102 and the interrogation device 104, the sensing device 102 operates as a passive NFC device. In this configuration, the sensing device 102 does not consume power from internal power source, such as the sensor power supply 144, for communication with the interrogation device 104. Instead, when interrogated by the interrogation device 104, the sensing device 102 can be powered by electromagnetic induction from magnetic fields produced near the interrogation device 104. However, it is recognized that, during the communication with the interrogation device 104, the sensing device 102 can be powered in different manners.

The processing unit 140 operates to control the sensor unit 132 and other components in the sensing device 102. An example of the processing unit 140 includes a central processing unit (CPU). Further, the processing unit 140 operates to communicate with the interrogation device 104. In some examples, the processing unit 140 receives signals from the antenna 136. In some examples, a demodulator is provided to demodulate an RF signal received via the antenna 136. The demodulator can be implemented in a way known in the art, including, for example, attenuator stage and amplifier stage. The processing unit 140 can perform various operations and generate an output signal for transmission. In some examples, a modulator is provided to modulate an output signal generated by the processing unit 140. The modulated signal is transmitted through the antenna 136 to the interrogation device 104. The modulator can be implemented in a way known in the art, including, for example, driver stage and amplifier stage. The processing unit 140 can be implemented in a way known in the art, including, for example, a processor, a decoder, and an encoder.

The storage unit 142 includes one or more memories configured to store the sensing device data 106. As described herein, the sensing device data 106 can contain physiological parameter data (e.g., measurement data) obtained from the sensor unit 132 and other data associated with the sensing device 102 and/or the subject S. The sensing device data 106 is further described with respect to FIG. 4. At least a portion of the sensing device data 106 is transmitted to and readable by the interrogation device 104. The storage unit 142 can be of various types, including volatile and nonvolatile, removable and non-removable, and/or persistent media. In some embodiments, the storage unit 142 is an erasable programmable read only memory (EPROM).

The sensor power supply 144 is included in the sensing device 102 and provides power to operate the sensor unit 132 and associated elements, such as the processing unit 140 and the storage unit 142. In some examples, the sensor power supply 144 includes one or more batteries, which is either for single use or rechargeable.

The heat isolation devices 150 operate to thermally isolate the sensor unit 132 from heat sources in the sensing device 102 and improve accuracy of physiological parameter readings by the sensor unit 132. As described herein, the sensing device 102 (e.g., a circuit board thereof) includes various components that can function as heat sources. In some embodiments, such heat sources in the sensing device 102 include a processor (such as a processing unit 140 in FIG. 3). In addition, the heat sources can be generated when the sensing device 102 is powered. For example, heat is generated by electromagnetic induction from magnetic fields that are produced as the interrogation device 104 is positioned close to the sensing device 102 for powering the sensing device 102.

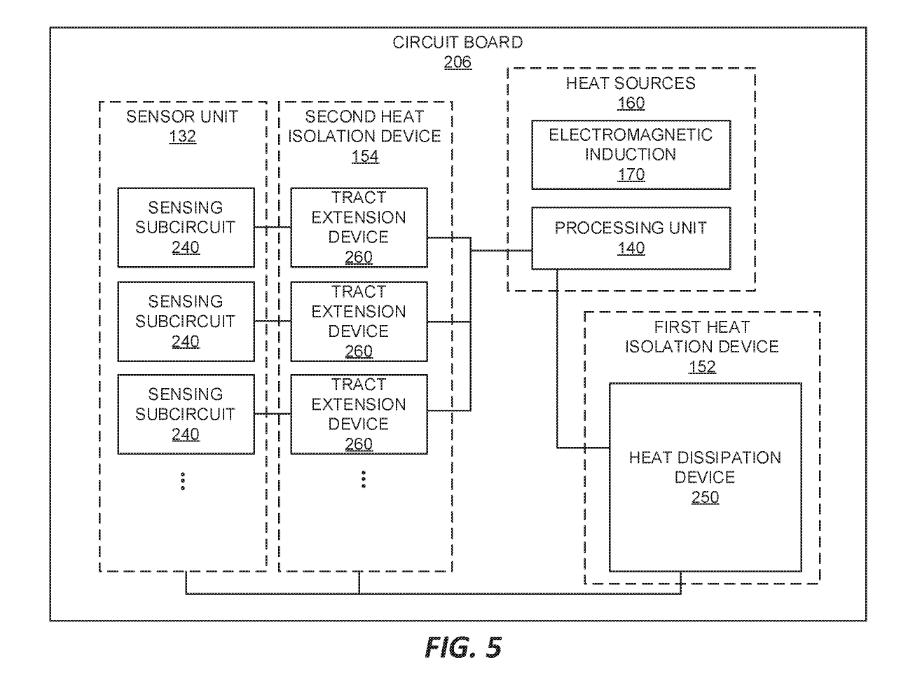

Heat from heat sources can cause undesirable effects on the operation of the sensor unit 132. For example, where the sensor unit 132 includes one or more temperature sensors such as thermistors, the heat sources can influence measurements of the temperature sensors, causing inaccurate temperature readings by the temperature sensors. The heat isolation devices 150 are configured to reduce the undesired effect of the heat sources on the temperature reading. Examples of the heat isolation devices 150 are further described with reference to FIG. 5.

FIG. 3 is a schematic, exploded view of the sensing device 102 configured as a temperature sensing patch. In the illustrated example, the sensing device 102 includes a cover 202, a binding device 204, a circuit board 206, a first insulator 208, a second insulator 210, and a mounting layer 212.

In this example, the sensing device 102 is configured to determine or predict a core temperature of a subject S. In some embodiments, the sensing device 102 includes a plurality of thermistors as the sensor unit 132, which are arranged with respect to the skin of a subject and operate to perform heat flux measurement. Example principles, configurations, and operations of the sensing device 102 as a core temperature sensing patch are described in U.S. Pat. No. 8,657,758, the disclosure of which is incorporated herein by reference in its entirety.

As described herein, the sensing device 102 includes various heat sources that may have an influence on operation of the thermistors, thereby causing inaccurate temperature readings. The sensing device 102 includes various features that reduce or minimize the effect of the heat sources on the temperature readings.

In some embodiments, the circuit board 206 is configured as a flexible printed circuit board that includes at least some of the components of the sensing device 102 as described with reference to FIG. 2. In some embodiments, the circuit board 206 includes a plurality of thermistors, such as four thermistors, and is flexed to arrange the plurality of thermistors in predetermined location in the sensing device 102. An example arrangement of the thermistors is described with reference to FIG. 4. An example configuration of the circuit board 206 is further described with reference to FIGS. 5-9.

In some embodiments, the circuit board 206 is disposed between the first insulator 208 and the second insulator 210. The cover 202 can be disposed on the first insulator 208, and the mounting layer 212 can be disposed on the second insulator 210, so that the mounting layer 212 is arranged opposite to the cover 202. The mounting layer 212 is configured to removably mount or attach the sensing device 102 on the subject. For example, the mounting layer 212 can be attached to, and thus directly contact with, the skin of the subject. In this example, the mounting layer 212 provides a mounting portion of the sensing device 102 that engages the sensing device 102 with the subject. In some embodiments, the mounting layer 212 includes an adhesive layer. In other embodiments, other portions of the sensing device 102 can be used as the mounting portion that engages the sensing device 102 to the subject. In yet other embodiments, a separate mounting device, such as a fastener, can be used with the sensing device 102 to mount the sensing device 102 to the subject.

The binding device 204 is used to assemble and hold the elements together, such as the cover 202, the circuit board 206, the first and second insulators 208 and 210, and the mounting layer 212. In some embodiments, the binding device 204 is configured as a woven material.

FIG. 4 schematically illustrates an example arrangement of thermistors for heat flux measurement. In this example, a temperature sensing patch as the sensing device 102 includes four thermistors 220, 222, 224, and 226. Three of the thermistors, such as a first thermistor 220, a second thermistor 222, and a fourth thermistor 226, are arranged adjacent to the skin 230 of the subject, and the other thermistor, such as a third thermistor 224, is arranged apart from the subject skin. In the illustrated example, the first, second, and fourth thermistors 220, 222, and 226 are disposed on the mounting layer 212 (e.g., an adhesive layer) so that the mounting layer 212 is disposed between the thermistors 220, 222, and 226 and the subject skin 230. In other examples, the first, second, and fourth thermistors 220, 222, and 226 are arranged to contact directly with the skin 230. The first, second, and fourth thermistors 220, 222, and 226 are arranged to be thermally isolated from the rest of the circuit board 206 so that the measurements are accurately obtained as skin temperatures without influence of heat sources in the circuit board 206.

The third thermistor 224 is disposed on an insulator, either or both of the first insulator 208 and the second insulator 210 and below the cover 202. The third thermistor 224 is arranged to be thermally isolated from ambient and the other thermistors.

In this configuration, the three thermistors arranged adjacent to the skin are used to measure heat flux from the skin thereunder, and the thermistor arranged oppositely to the three thermistors is used to measure heat flux away from the skin. The measured heat fluxes are used to determine how much heat the body is losing or how insulting the skin is being. Further, a core temperature of the subject can be calculated based on the determination. Example methods of determining a core temperature is further described in U.S. Pat. No. 8,657,758, the disclosure of which is incorporated herein by reference in its entirety.

FIG. 5 is a block diagram of the circuit board 206. As described herein, the circuit board 206 includes the sensor unit 132, the processing unit 140, and the heat isolation device 150.

In some embodiments, the processing unit 140 can be a heat source 160 during operation. The heat source 160 generates heat and can influence operations of other components of the circuit board 206, such as the sensor unit 132. For example, the heat generated from the heat source 160 can affect the measurements that are performed by the sensing subcircuits 240 and thus cause inaccurate readings. In addition, the heat source 160 can be created by electromagnetic induction 170 in electromagnetic field produced when the interrogation device 104 is located proximate to the sensing device 102 for powering the sensing device 102. Other heat sources are also possible in other embodiments.

The sensor unit 132 includes one or more sensing subcircuits 240. Each sensing subcircuit 240 is configured to implement a sensor in the sensor unit 132. Where the sensing device 102 is a temperature sensing patch, the sensing subcircuits 240 can be configured to include a thermistor.

The heat isolation device 150 includes a plurality of heat isolation devices, such as a first heat isolation device 152 and a second heat isolation device 154. The first heat isolation device 152 includes a heat dissipation device 250. The second heat isolation device 154 includes one or more track extension devices 260.

The heat dissipation device 250 is configured to dissipate heat from one or more heat sources in the circuit board 206. The heat dissipation device 250 can also serve as a heat storage device, by having a large thermal mass that takes a long time to rise in temperature. The heat dissipates while the sensor unit 132 is not actively sensing, enabling the heat dissipation device 250 to cool off and absorb more heat the next time the sensor unit 132 is in use. As described herein, the processing unit 140 functions as a heat source in operation. Other components in the sensing device 102 can also work as heat sources.

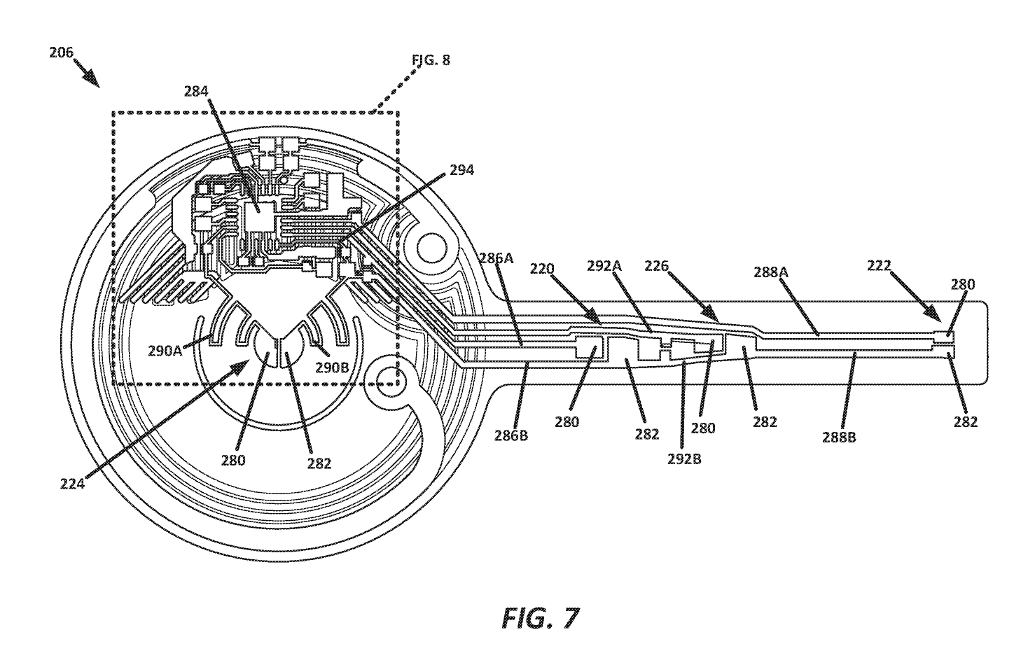

In this example, the heat dissipation device 250 is connected to the processing unit 140 to draw and dissipate heat generated from the processing unit 140 and thus prevent or reduce the heat that travels from the processing unit 140 to the sensor unit 132. The heat dissipation device 250 is made in various configurations. An example of the heat dissipation device 250 is illustrated in FIG. 7.

The track extension device 260 is associated with the sensing subcircuit 240 and configured to reduce influence of the heat sources on the sensing subcircuit 240. In addition, the track extension device 260 is configured to reduce electromagnetic interference (EMI) that can be caused in electromagnetic fields generated by the interrogation device 104 with respect to the sensing device 102. In some examples, the track extension device 260 includes one or more conductive tracks routed between the sensing subcircuit 240 and the heat sources (e.g., the processing unit) and configured to reduce undesired effect of the heat sources and/or electromagnetic interference. For example, the conductive track of the track extension device is configured to be thin and routed in a long path between the processing unit and the sensing subcircuit. An example of the track extension device 260 is further described and illustrated in FIG. 7.

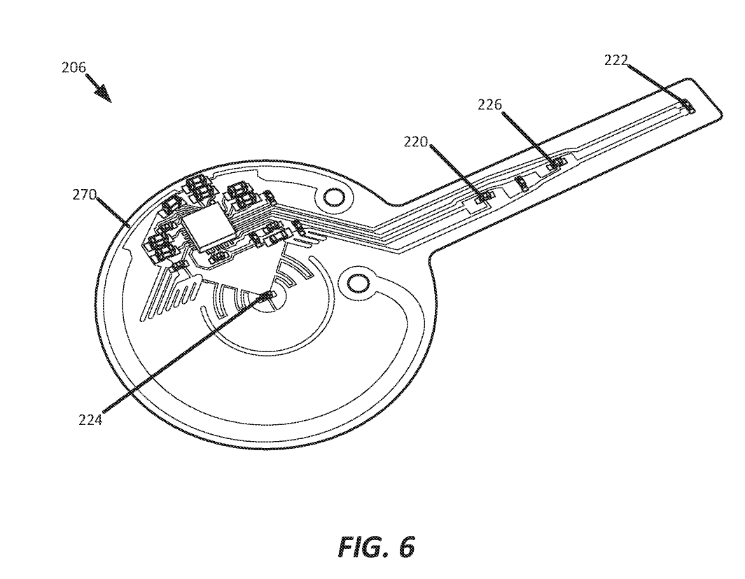

FIG. 6 schematically illustrates an example of the circuit board 206. In this example, the circuit board 206 is configured for a temperature sensing patch and includes four thermistors 220, 222, 224, and 226. In some embodiments, the circuit board 206 is configured as a flexible printed circuit board (PCB) 270 and includes at least some of the components of the sensing device 102 as described with reference to FIG. 2.

FIG. 7 is a schematic view of a front side of the circuit board 206 of FIG. 6, which illustrates conductive tracks and pads. As illustrated, the circuit board 206 is used to mechanically support and electrically connect electronic components, such as illustrated in FIG. 2, using the conductive tracks and pads. In some embodiments, the conductive tracks and pads are etched from metal sheets (e.g., copper sheets) laminated onto a non-conductive substrate. Various components such as those illustrated in FIG. 2 can be soldered on or embedded in the circuit board 206.

Referring to FIG. 7, each of the thermistors 220, 222, 224, and 226 can include a pair of thermistor pads 280 and 282 which are closely arranged and space apart from each other on the circuit board 206. In some embodiments, the thermistor pads 280 and 282 are configured to have enlarged areas so that the thermistor pads collect heat from the subject body and reach a stable temperature as quickly as possible.

Each of thermistor pads 280 and 282 is electrically connected to the processing unit 140 at a processing unit installation area 284. In the illustrated example, a first thermistor track 286A is routed on the circuit board 206 to connect between the processing unit installation area 284 and a first thermistor pad 280 of the first thermistor 220, and a second thermistor track 286B is routed on the circuit board 206 to connect between the processing unit installation area 284 and a second thermistor pad 282 of the first thermistor 220. A first thermistor track 288A is routed on the circuit board 206 to connect between the processing unit installation area 284 and a first thermistor pad 280 of the second thermistor 222, and a second thermistor track 288B is routed on the circuit board 206 to connect between the processing unit installation area 284 and a second thermistor pad 282 of the second thermistor 222. A first thermistor track 290A is routed on the circuit board 206 to connect between the processing unit installation area 284 and a first thermistor pad 280 of the third thermistor 224, and a second thermistor track 290B is routed on the circuit board 206 to connect between the processing unit installation area 284 and a second thermistor pad 282 of the third thermistor 224. A first thermistor track 292A is routed on the circuit board 206 to connect between the processing unit installation area 284 and a first thermistor pad 280 of the fourth thermistor 226, and a second thermistor track 292B is routed on the circuit board 206 to connect between the processing unit installation area 284 and a second thermistor pad 282 of the fourth thermistor 226.

In some embodiments, at least some of the first thermistor tracks 286A, 288A, 290A, and 292A can be routed to partially overlap each other. In other embodiments, at least some of the second thermistor tracks 286B, 288B, 290B, and 292B can be routed to partially overlap each other. In the illustrated example, the second thermistor tracks 286B, 288B, 290B, and 292B are branched out from a single track 294 extending from the processing unit installation area 284. Other various configurations are also possible.

As described herein, at least one of the first thermistor tracks 286A, 288A, 290A, and 292A is configured as a thin trace to reduce heat that is conducted from the heat source 160 to the first thermistor pads 280 of the thermistors 220, 222, 224, and 226. In some embodiments, the first thermistor track 286A, 288A, 290A, 292A can have a width smaller than a width of the corresponding first thermistor pad 280. In other embodiments, the width of the first thermistor track 286A, 288A, 290A, 292A can range from about 5 mil to about 25 mil. In yet other embodiments, the width of the first thermistor track 286A, 288A, 290A, 292A can range from about 8 mil to about 18 mil. In yet other embodiments, the width of the first thermistor track 286A, 288A, 290A, 292A can be smaller than 5 mil.

Similarly, at least one of the second thermistor tracks 286B, 288B, 290B, and 292B is configured as a thin trace to reduce heat that is conducted from the heat source 160 to the second thermistor pads 280 of the thermistors 220, 222, 224, and 226. In some embodiments, the second thermistor track 286B, 288B, 290B, 292B can have a width smaller than a width of the corresponding second thermistor pad 282. In other embodiments, the width of the second thermistor track 286B, 288B, 290B, 292B can range from about 5 mil to about 25 mil. In yet other embodiments, the width of the second thermistor track 286B, 288B, 290B, 292B can range from about 8 mil to about 18 mil. In yet other embodiments, the width of the second thermistor track 286B, 288B, 290B, 292B can be smaller than 5 mil.

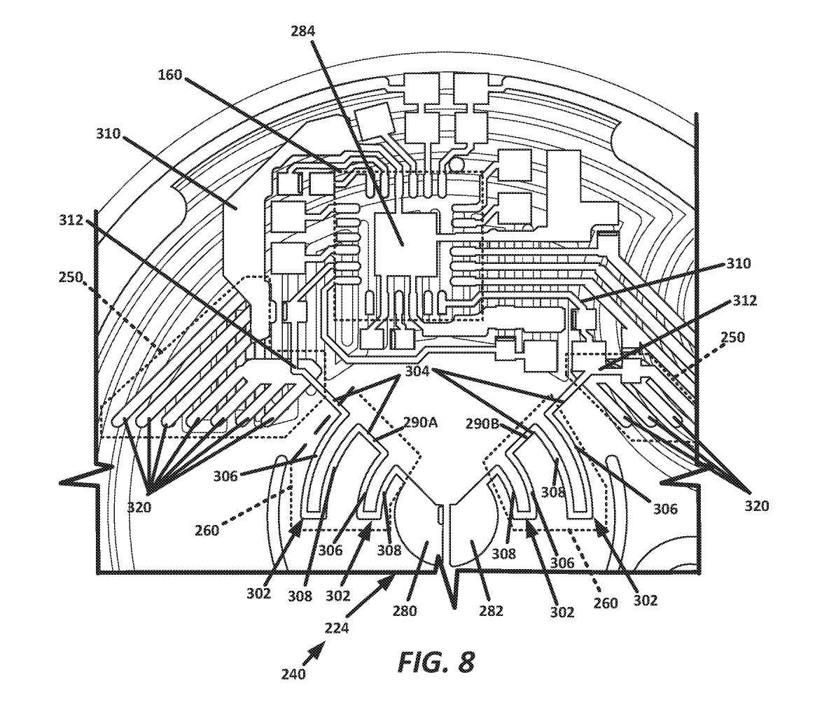

FIG. 8 is an expanded view of the circuit board 206 of FIG. 7. As generally described in FIG. 5, the circuit board 206 includes the heat source 160, the heat dissipation devices 250, and the track extension devices 260. In this example, the heat source 160 includes the processing unit installation area 284 at which the processing unit 140 is mounted.

The sensing subcircuits 240, which include the thermistor pads, are arranged to be thermally isolated from the heat source 160 so that the operation of sensing subcircutis 240 are not influenced by heat generated by the heat source 160. In the illustrated example, the first and second thermistor pads 280 and 282 of the third thermistor 224 are arranged apart from the heat source 160 with the track extension devices 260 therebetween.

The track extension devices 260 include the thermistor tracks 290A and 290B that electrically connect the thermistor pads 280 and 282 to the processing unit 140. The track extension devices 260 operate to reduce undesired effect from the heat source 160. In addition, the track extension devices 260 are configured to prevent crosstalk between adjacent thermistor pads and/or between adjacent thermistors. Further, the track extension devices 260 can be configured to reduce wireless communication interference, such as RFID interference or NFC interference.

As illustrated in FIG. 8, the first thermistor track 290A is routed between the first thermistor pad 280 and the processing unit installation area 284. In some embodiments, the first thermistor track 290A is configured as a thin trace to reduce heat conduction from the heat source 160 to the first thermistor pad 280. In some embodiments, the first thermistor track 290A has a width smaller than a width of the connected first thermistor pad 280. In other embodiments, the width of the first thermistor track 290A can range from about 5 mil to about 25 mil. In yet other embodiments, the width of the first thermistor track 290A can range from about 8 mil to about 18 mil. In yet other embodiments, the width of the first thermistor track 290A can be smaller than 5 mil. Although the first thermistor track is made to be thin, the thickness or width of the first thermistor track needs to be thick enough not to influence the resistance reading on the thermistor, and thick enough to be printed using affordable processes.

In some embodiments, the first thermistor track 290A is routed in a loop, thereby lengthening an entire length of the first thermistor track 290A. The first thermistor track 290A is routed such that a length of the first thermistor track 290A is longer than a distance between the first thermistor pad 280 and the processing unit installation area 284. In some examples, a ratio can range from about 2:1 to about 10:1 between the length of the first thermistor track 290A and the distance from the first thermistor pad 280 to the processing unit installation area 284. Other ratios are also possible to the extent that the length of the first thermistor track 290A is longer than the distance between the first thermistor pad 280 and the processing unit installation area 284.

In some embodiments, the track extension device 260 includes one or more stretch-out portions 302 in which the first thermistor track 290A is stretched out from a main track 304, thereby increasing the length of the first thermistor track 290A. The extended length of thin thermistor tracks can reduce or prevent not only crosstalk between adjacent thermistor pads and/or between adjacent thermistors, but undesired effect from the heat sources in the circuit.

The stretch-out portion 302 can be of various configurations. In some embodiments, the stretch-out portion 302 is configured to form a pair of track lines 306 and 308 that extend out from the main track 304. The pair of track lines 306 and 308 can be routed to be parallel with each other. The parallel track lines 306 and 308 can cancel a current or voltage induced in or with respect to one of the paired track lines in an electromagnetic field with a current or voltage induced in or with respect to the other track line in the electromagnetic field. Thus, the parallel track lines can prevent wireless communication interference, such as RFID interference or NFC interference. In some embodiments, the track lines 306 and 308 of the stretch-out portion 302 can be curved around the first thermistor pad 280. In other embodiments, the stretch-out portion 302 can have other patterns, such as a zig-zag, a wave, a serpentine shape, or a spiral shape.

The second thermistor track 290B can be routed similarly to the first thermistor track 290A. The second thermistor track 290B can have the track extension device 260 similarly to the first thermistor track 290A as described above. For example, the second thermistor track 290B is routed in a loop, thereby lengthening an entire length of the second thermistor track 290B. The second thermistor track 290B can have a length longer than a distance between the second thermistor pad 282 and the processing unit installation area 284. A ratio between the length of the second thermistor track 290B and the distance from the second thermistor pad 282 to the processing unit installation area 284 can range between about 2:1 to about 10:1. The stretch-out portion 302 of the second thermistor track 290B can be configured and arranged similarly to the stretch-out portion 302 of the first thermistor track 290A. In some embodiments, the track extension device 260 of the second thermistor track 290B can be configured and arranged symmetrically to the track extension device 260 of the first thermistor track 290A about a hypothetical line extending between the first thermistor pad 280 and the second thermistor pad 282.

Although the track extension devices 260 is primarily described with respect to the third thermistor 224, other thermistors, such as the first, second, and fourth thermistors 220, 222, and 226, can have the track extension devices 260 in the same or similar configurations and arrangements.

Referring still to FIG. 8, the heat dissipation device 250 operates to dissipate heat from the heat source 160 in the circuit board 206. In the illustrated example, the processing unit 140 functions as a primary heat source 160 when the processing unit 140 is in operation. Other heat sources can generate heat in the circuit board 206, such as electromagnetic induction or other components in the sensing device 102.

In this example, the heat dissipation device 250 is connected to the processing unit 140 through a connecting trace 310 and thus draws heat from the processing unit 140 by heat conduction. In addition, the heat dissipation device 250 can also be connected to the thermistor track 290A, 290B through a connecting trace 312, and thus prevent the heat source 160 (e.g., the processing unit 140 in this example) from transmitting heat down to the thermistor pads 280, 282.

In some embodiments, the heat dissipation device 250 is arranged close from the heat source 160 to fast and effective dissipation of heat from the heat source 160. In addition or alternatively, the heat dissipation device 250 is arranged away from the thermistor pads 280 and 282 to prevent influence of heat on the thermistor pads 280 and 282. In other embodiments, the heat dissipation device 250 is arranged adjacent an edge of the circuit board 206 to improve heat removal to the surroundings.

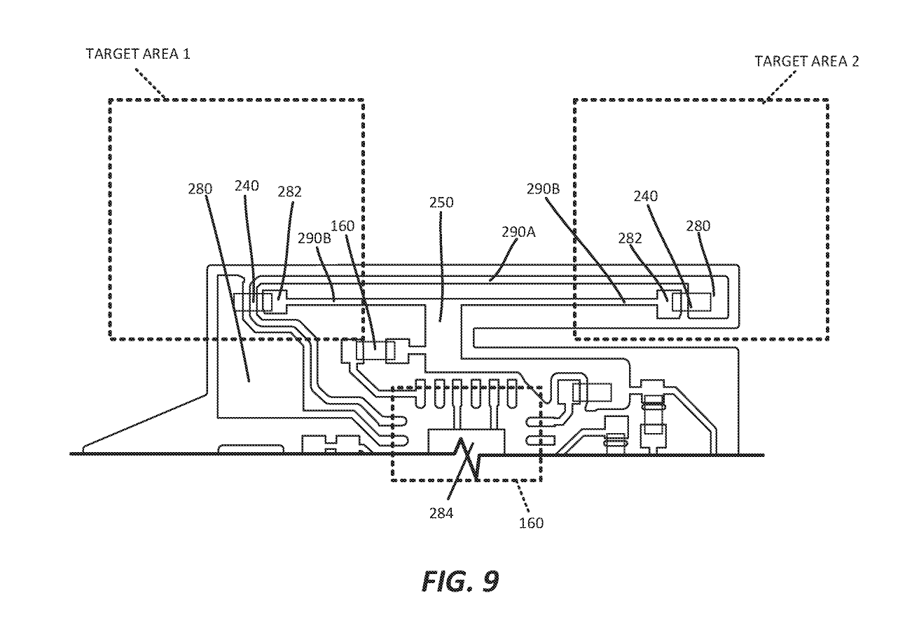

The heat dissipation device 250 can be of various configurations. In some embodiments, as illustrated in FIG. 8, the heat dissipation device 250 includes a plurality of trace prongs 320. The trace prongs 320 can be arranged to project in one or more directions away from the heat source 160. At least some of the trace prongs 320 can extend in the same direction. Alternatively, the trace prongs 320 extend different directions. In other embodiments, the heat dissipation device 250 is configured as a solid conductive pad, such as illustrated in FIG. 9. Other configurations of the heat dissipation device 250 are also possible in yet other embodiments.

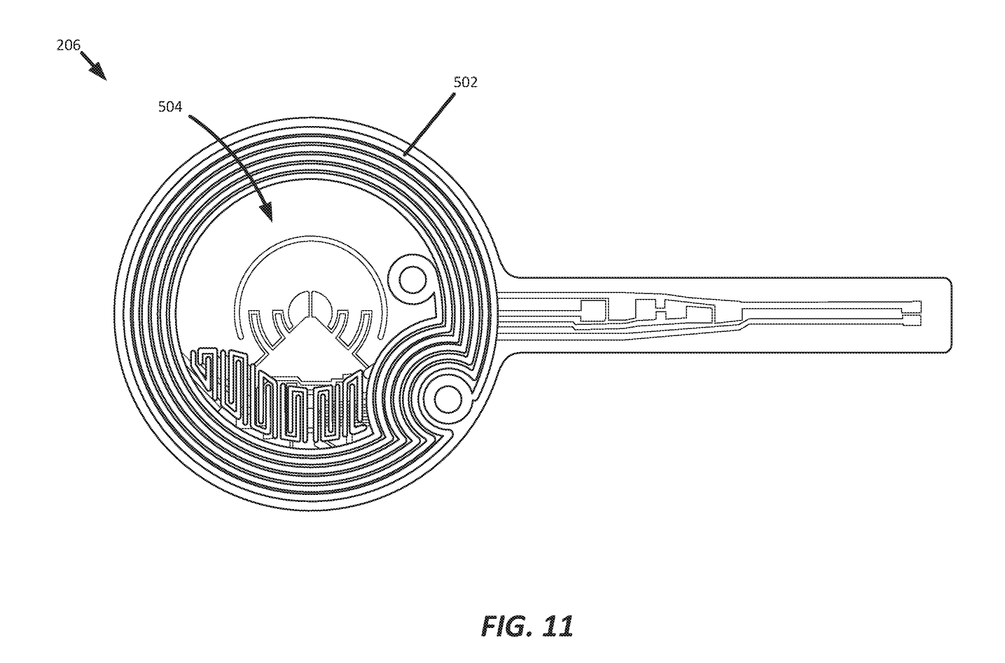

In some embodiments, as illustrated in FIG. 11, the rear side of the circuit board 206 includes a copper-fill 502. When the copper-fill 502 is exposed to the electromagnetic field (e.g., NFC field), any solid or circular shape may develop eddy current heat that affects the temperature readings and decreases the efficiency of the antenna. To address this, the shape of the copper-fill 502 can be configured to have no closed loops in order to prevent eddy currents and provide heat buffer and/or dissipation for the heat source (e.g., the CPU). In addition, the copper-fill 502 can be arranged away from the location of the thermistor pads (e.g., the third thermistor 224 at the center of the circular part of the board) to prevent any heat cross-talk. An interior portion 504 forms other components, such as shielding and heat sink areas. Other configurations are possible.

FIG. 9 is a partial schematic view of another example circuit board 206, which illustrates conductive tracks and pads. Similarly to the example shown in FIG. 8, the circuit board 206 in this example includes a plurality of thermistors, each including a pair of thermistor pads 280 and 282. The circuit board 206 includes one or more heat sources 160 at or around the processing unit installation area 284. The thermistor pads 280 and 282 are connected to the processing unit installation area 284 through thermistor tracks 290A and 290B. The thermistor tracks 290A and 290B are configured as thin traces and routed in a loop to extend the length of the thermistor tracks. The circuit board 206 further includes a heat dissipation device 250. The configurations and arrangements of these components of the circuit board 206 are identical or similar to those described herein, and thus the detailed description thereof is omitted for brevity purposes.

FIG. 10 illustrates an exemplary architecture of the interrogation device 104. The interrogation device 104 illustrated in FIG. 10 is used to execute the operating system, application programs, and software modules (including the software engines) described herein.

The interrogation device 104 is a computing device of various types. In some embodiments, the interrogation device 104 is a mobile computing device. Examples of the interrogation device 104 as a mobile computing device include a mobile device (e.g., a smart phone and a tablet computer), a wearable computer (e.g., a smartwatch and a head-mounted display), a personal digital assistant (PDA), a handheld game console, a portable media player, a ultra-mobile PC, a digital still camera, a digital video camera, and other mobile devices. In other embodiments, the interrogation device 104 is other computing devices, such as a desktop computer, a laptop computer, or other devices configured to process digital instructions.

It is recognized that the architecture illustrated in FIG. 10 can also be implemented in other computing devices used to achieve aspects of the present disclosure. For example, the data management system 110 can be configured similarly to the architecture of FIG. 10. To avoid undue repetition, this description of the interrogation device 104 will not be separately repeated herein for each of the other computing devices including the data management system 110.

The interrogation device 104 includes, in some embodiments, at least one processing device 402, such as a central processing unit (CPU). A variety of processing devices are available from a variety of manufacturers, for example, Intel or Advanced Micro Devices. In this example, the interrogation device 104 also includes a system memory 404, and a system bus 406 that couples various system components including the system memory 404 to the processing device 402. The system bus 406 is one of any number of types of bus structures including a memory bus, or memory controller; a peripheral bus; and a local bus using any of a variety of bus architectures.

The system memory 404 includes read only memory 408 and random access memory 410. A basic input/output system 412 containing the basic routines that act to transfer information within the interrogation device 104, such as during start up, is typically stored in the read only memory 408.

The interrogation device 104 also includes a secondary storage device 414 in some embodiments, such as a hard disk drive, for storing digital data. The secondary storage device 414 is connected to the system bus 406 by a secondary storage interface 416. The secondary storage devices and their associated computer readable media provide nonvolatile storage of computer readable instructions (including application programs and program modules), data structures, and other data for the interrogation device 104.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media.

A number of program modules can be stored in secondary storage device 414 or memory 404, including an operating system 418, one or more application programs 420, other program modules 422, and program data 424.

In some embodiments, the interrogation device 104 includes input devices to enable a user to provide inputs to the interrogation device 104. Examples of input devices 426 include a keyboard 428, a pointer input device 430, a microphone 432, and a touch sensitive display 440. Other embodiments include other input devices. The input devices are often connected to the processing device 402 through an input/output interface 438 that is coupled to the system bus 406. These input devices 426 can be connected by any number of input/output interfaces, such as a parallel port, serial port, game port, or a universal serial bus. Wireless communication between input devices and interface 438 is possible as well, and includes infrared, BLUETOOTH® wireless technology, 802.11a/b/g/n, cellular, or other radio frequency communication systems in some possible embodiments.

In this example embodiment, a touch sensitive display device 440 is also connected to the system bus 406 via an interface, such as a video adapter 442. The touch sensitive display device 440 includes touch sensors for receiving input from a user when the user touches the display. Such sensors can be capacitive sensors, pressure sensors, or other touch sensors. The sensors not only detect contact with the display, but also the location of the contact and movement of the contact over time. For example, a user can move a finger or stylus across the screen to provide written inputs. The written inputs are evaluated and, in some embodiments, converted into text inputs.

In addition to the display device 440, the interrogation device 104 can include various other peripheral devices (not shown), such as speakers or a printer.

The computing device 400 further includes a communication device 446 configured to establish communication across the network. In some embodiments, when used in a local area networking environment or a wide area networking environment (such as the Internet), the computing device 400 is typically connected to the network through a network interface, such as a wireless network interface 448. Other possible embodiments use other wired and/or wireless communication devices. For example, some embodiments of the computing device 400 include an Ethernet network interface, or a modem for communicating across the network. In yet other embodiments, the communication device 446 is capable of short-range wireless communication. Short-range wireless communication is one-way or two-way short-range to medium-range wireless communication. Short-range wireless communication can be established according to various technologies and protocols. Examples of short-range wireless communication include a radio frequency identification (RFID), a near field communication (NFC), a Bluetooth technology, and a Wi-Fi technology.

The interrogation device 104 typically includes at least some form of computer-readable media. Computer readable media includes any available media that can be accessed by the interrogation device 104. By way of example, computer-readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the interrogation device 104. Computer readable storage media does not include computer readable communication media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 10 is also an example of programmable electronics, which may include one or more such computing devices, and when multiple computing devices are included, such computing devices can be coupled together with a suitable data communication network so as to collectively perform the various functions, methods, or operations disclosed herein.

Referring again to FIG. 10, the interrogation device 104 can include a location identification device 450. The location identification device 450 is configured to identify the location or geolocation of the interrogation device 104. The location identification device 450 can use various types of geolocating or positioning systems, such as network-based systems, handset-based systems, SIM-based systems, Wi-Fi positioning systems, and hybrid positioning systems. Network-based systems utilize service provider's network infrastructure, such as cell tower triangulation. Handset-based systems typically use the Global Positioning System (GPS). Wi-Fi positioning systems can be used when GPS is inadequate due to various causes including multipath and signal blockage indoors. Hybrid positioning systems use a combination of network-based and handset-based technologies for location determination, such as Assisted GPS.

The various examples and teachings described above are provided by way of illustration only and should not be construed to limit the scope of the present disclosure. Those skilled in the art will readily recognize various modifications and changes that may be made without following the examples and applications illustrated and described herein, and without departing from the true spirit and scope of the present disclosure.

What is claimed is:

1. A physiological parameter sensing device, the sensing device comprising:
a mounting portion configured to engage with a subject; and
a circuit board including:
a processing unit;
a sensing subcircuit connected to the processing unit and configured to measure a physiological parameter of the subject; and
a heat isolation device connected to the processing unit and configured to thermally isolate the sensing subcircuit from the processing unit.

2. The physiological parameter sensing device of claim 1, wherein the heat isolation device includes a heat dissipation device connected to the processing unit and draw dissipate heat generated from the processing unit.

3. The physiological parameter sensing device of claim 2, wherein the heat dissipation device includes a plurality of trace prongs.

4. The physiological parameter sensing device of claim 1, wherein the heat isolation device includes a track extension device having a conductive track routed between the sensing subcircuit and the processing unit.

5. The physiological parameter sensing device of claim 4, wherein the conductive track of the track extension device has a width ranging from about 5 mil to about 25 mil.

6. The physiological parameter sensing device of claim 4, wherein the conductive track of the track extension device is routed in a loop to make a length of the conductive track longer than a distance between the processing unit and the sensing subcircuit.

7. The physiological parameter sensing device of claim 4, wherein the track extension device includes a stretch-out portion, the stretch-out portion having a pair of track lines extending out from a main track.

8. The physiological parameter sensing device of claim 7, wherein the pair of track lines are arranged to be parallel with each other.

9. The physiological parameter sensing device of claim 1, wherein the sensing subcircuit includes a thermistor pad.

10. The physiological parameter sensing device of claim 9, wherein the heat isolation device includes a track extension device having a conductive track routed between the sensing subcircuit and the processing unit, the conductive track having a width smaller than a width of the thermistor pad.

11. A temperature sensing patch comprising:
a mounting layer engagable with a subject; and
a circuit board including:
a processing unit;
a pair of thermistor pads configured to measure heat flux associated with the subject;
a pair of thermistor tracks connecting the pair of thermistor pads to the processing unit; and
a heat isolation device connected to the processing unit and configured to thermally isolate the pair of thermistor pads from the processing unit.

12. The temperature sensing patch of claim 11, wherein the heat isolation device includes a heat dissipation device connected to the processing unit and draw dissipate heat generated from the processing unit.

13. The temperature sensing patch of claim 12, wherein the heat dissipation device includes a plurality of trace prongs.

14. The temperature sensing patch of claim 11, wherein at least a portion of the pair of thermistor tracks has a width smaller than a width of either of the pair of thermistor pads.

15. The temperature sensing patch of claim 11, wherein at least a portion of the pair of thermistor tracks has a width ranging from 5 mil to about 25 mil.

16. The temperature sensing patch of claim 11, wherein the pair of thermistor tracks is routed in a loop to make a length of the pair of thermistor tracks longer than a distance between the processing unit and the pair of thermistor pads.

17. The temperature sensing patch of claim 4, wherein the pair of thermistor tracks includes a stretch-out portion, the stretch-out portion having a pair of track lines extending out from a main track.

18. The temperature sensing patch of claim 17, wherein the pair of track lines are arranged to be parallel with each other.

19. A system for monitoring a physiological parameter of a subject, the system comprising:
a sensing device including:
a mounting portion engaged with a subject; and
a circuit board including:
a processing unit;
a sensing subcircuit connected to the processing unit and configured to measure a physiological parameter of the subject; and
a heat isolation device connected to the processing unit and configured to thermally isolate the sensing subcircuit from the processing unit; and
an interrogation device operable by a user and configured to:
establish communication with the sensing device; and
receive measurement data from the sensing device.

20. The system of claim 19, wherein the interrogation device is configured to communicate with the sensing device via short range wireless communication.
